(19) United States
(12) Patent Application Publication
(10) Pub. No.: US 2025/0186313 A1
(43) Pub. Date: Jun. 12, 2025

(54) SCALP OR HAIR CARE COMPOSITION COMPRISING NANO-CARBON STRUCTURE

(71) Applicant: INBCT Co., Ltd., Seoul (KR)

(72) Inventors: Jaechul RYU, Uiwang-si (KR); Soon Won CHOI, Anyang-si (KR)

(73) Assignee: INBCT Co., Ltd., Seoul (KR)

(21) Appl. No.: 18/847,202

(22) PCT Filed: Mar. 15, 2023
(86) PCT No.: PCT/KR2023/003466

§ 371 Date: Sep. 13, 2024

(30) Foreign Application Priority Data
Mar. 15, 2022 (KR) 10-2022-0032266

(51) Int. Cl. A61K 8/19; A61K 8/02; A61K 8/86; A61K 33/44; A61Q 5/00

(52) U.S. Cl. CPC A61K 8/19; A61K 8/0241; A61K 8/86; A61K 33/44; A61Q 5/006

(57) ABSTRACT

Provided is a scalp or hair care composition including a nano-carbon structure. The nano-carbon structure can retain moisture of the scalp or hair even in a small amount, and relieve inflammation of the scalp to increase a hair and scalp care effect. In addition, the nano-carbon structure has the advantage of being applicable to various mixtures due to maximized dispersibility.

### TECHNICAL FIELD

This application claims priority to and the benefit of Korean Patent Application No. 10-2022-0032266, filed on Mar. 15, 2022, the disclosure of which is incorporated herein by reference in its entirety.

The present invention relates to a composition for scalp or hair care including a nano-carbon structure, and more specifically to a composition for scalp or hair care including nano-graphene oxide, a nano-graphene oxide variant or graphene quantum dots, a pharmaceutical composition for preventing and treating a scalp disease, a method for treating a scalp disease and the use of a nano-carbon structure in the treatment of a scalp disease.

### BACKGROUND ART

The scalp is skin tissue that surrounds the surface of the head, responds to external friction and protects the inside of the scalp from the external environment. The scalp is frequently exposed to skin-harmful rays such as ultraviolet rays, chemical products for beauty and various changes in the external environment, and the hair cuticle layer, hair roots and pores can be easily damaged due to various physical stimuli such as pulling on the hair, and this can develop into scalp diseases such as inflammation.

The majority of people feel that their skin or hair is in the best condition when their scalp or hair is adequately hydrated, and depending on the season, in spring or fall, when temperature and/or humidity changes rapidly, many people feel that their scalp or hair is in optimal condition. and feel that gloss is reduced. In addition, external stimuli such as environmental pollution, stress, nutritional or hormonal imbalances induce the excessive development of sebaceous glands distributed on the scalp or induce the excessive proliferation of keratinocytes and the occurrence of inflammatory reactions. These are the causes of scalp diseases such as psoriasis, seborrheic dermatitis and dandruff, which cause the symptoms of rash, erythema and itching, and worsen the health of the scalp, thereby resulting in hair thinning and hair loss. The main treatment for these scalp diseases is to relieve symptoms through consistent daily care, and thus, a medicated shampoo with excellent scalp moisture management and cleansing effects is needed. Generally, hair products with enhanced moisturizer ingredients are used to replenish the moisture and shine of the scalp and hair. Graphene is one of the allotropes of carbon and refers to a structure in which carbon atoms are gathered together to form a two-dimensional plane. Nano graphene is nano-sized graphene and is a type of nano-carbon structure. Nano graphene contains a large amount of oxygen, which makes up water, and can help maintain moisture in the scalp and hair and control moisture content, and thus, it has a moisturizing effect on its own. In addition, by utilizing electrical conductivity, which is one of the characteristics of graphene, the activity of the human body and hair cells can be increased by increasing the influence of microcurrents generated in the human body. Furthermore, graphene can alleviate changes in the scalp and hair condition caused by seasonal temperature and humidity changes. Recently, the effectiveness of nanocarbons in suppressing inflammatory responses was reported. In 2018, a British research team published research results showing that nano-sized graphene oxide inhibits the production of inflammatory cytokines, including IL-1 (ACS Nano 2018, 12, 12, 11949-11962). The inflammatory cytokines mentioned in this study are inflammatory cytokines that induce an immune response that causes symptoms of scalp diseases such as psoriasis, seborrheic dermatitis and dandruff, and it shows that nano-graphene oxide can be effectively utilized in the inflammatory response of scalp diseases.

Accordingly, as a result of making diligent efforts to develop a nano-carbon structure that has an excellent moisturizing effect on the scalp and hair and has anti-inflammatory and stratum corneum stabilizing effects in scalp cells, the present invention confirmed the anti-inflammatory effect of the nano-carbon structure on scalp diseases, and the present invention was completed by confirming that the aminated or PEGylated nano-carbon structure has much better moisturizing and anti-inflammatory effects on the scalp and hair than the nano-carbon structure itself and nano-carbon structures attached with various other functional groups, including oxygen.

### RELATED ART DOCUMENTS

### Patent Documents

(Patent Document 1) Korean Laid-Open Patent Publication No. 10-2021-0093746 (Jul. 16, 2021)

(Patent Document 2) Korean Laid-Open Patent Publication No. 10-2021-0119705 (Mar. 25, 2020)

### DISCLOSURE

### Technical Problem

An object of the present invention is to provide a composition for scalp or hair care including a nano-carbon structure, a pharmaceutical composition for preventing and treating a scalp disease, a method for treating a scalp disease and the use of a nano-carbon structure in the treatment of a scalp disease.

### Technical Solution

In order to achieve the above object of the present invention, provided is a composition for scalp or hair care, including a nano-carbon structure as an active ingredient.

In the present invention, the nano-carbon structure may be at least one selected from the group consisting of nano-graphene oxide, a nano-graphene oxide variant and graphene quantum dots.

In the present invention, the nano-graphene oxide variant may be an aminated nano-graphene oxide or a PEGylated nano-graphene oxide.

In the present invention, the nano-carbon structure may have an average diameter of 1 to 100 nm.

In the present invention, the nano-carbon structure may have a thickness of 0.1 to 5 nm.

In the present invention, the nano-carbon structure may be included at a concentration of 0.001 to 1,000 μg/mL.

In the present invention, the aminated nano-graphene oxide may include an amine functional group and a basic component of nano-graphene oxide at a ratio of 1:10,000 to 1:1.

In the present invention, the PEG functional group of the PEGylated nano-graphene oxide may be 4arm-PEG or 6arm-PEG.

In addition, the present invention provides a pharmaceutical composition for preventing and treating a scalp disease, including a nano-carbon structure as an active ingredient.

In the present invention, the nano-carbon structure may be at least one selected from the group consisting of nano-graphene oxide, a nano-graphene oxide variant and graphene quantum dots.

In the present invention, the nano-graphene oxide variant may be an aminated nano-graphene oxide or a PEGylated nano-graphene oxide.

In the present invention, the nano-carbon structure may have an average diameter of 1 to 100 nm.

In the present invention, the nano-carbon structure may have a thickness of 0.1 to 5 nm.

In the present invention, the nano-carbon structure may be included at a concentration of 0.001 to 1,000 μg/mL.

In the present invention, the aminated nano-graphene oxide may include an amine functional group and a basic component of nano-graphene oxide at a ratio of 1:10,000 to 1:1.

In the present invention, the PEG functional group of the PEGylated nano-graphene oxide may be 4arm-PEG or 6arm-PEG.

In the present invention, the scalp disease may be at least one selected from the group consisting of psoriasis, seborrheic dermatitis, dandruff and alopecia.

In addition, the present invention provides a method for treating a scalp disease, including the step of administering a nano-carbon structure to a patient with a scalp disease.

In addition, the present invention provides the use of a nano-carbon structure in the treatment of a scalp disease.

Unless otherwise defined, all technical and scientific terms used in the present specification have the same meanings as commonly understood by a person skilled in the art to which the present invention pertains. In general, the nomenclature used in the present specification is well known and commonly used in the art.

As used herein, the term “nano-carbon structure (nano-graphene oxide)” may refer to a carbon structure having nano-sized fragments by treating a carbon structure with various nanonization methods, and in the present invention, it may be a concept including nano-sized carbon graphene, or nano-sized graphene oxide (nano-GO; NGO) or graphene quantum dots (GQD) obtained by oxidizing the carbon graphene. In addition, it may include graphene particles having a width, length and height of several nanometers, which are prepared through appropriate processing. In the present invention, the nano-carbon structure may be particles having an average diameter of 10 to 50 nm and a thickness of 0.5 to 3 nm. In addition, with regard to the preparation method, carbon fiber may be obtained through thermo-oxidative cutting.

As used herein, the term “graphene oxide” may be abbreviated as “GO” and may include a structure in which a functional group containing an oxygen atom such as a carboxyl group, a hydroxy group or an epoxy group is bonded to graphene.

In the present invention, heat transfer occurs through the transfer medium of the crystal lattice of graphene formed through covalent bonds between carbon atoms. The thermal conductivity of the graphene is about 5,300 W/mk, which means that the thermal conductivity is very excellent. The graphene has an excellent ability to maintain the temperature of the hair and scalp and can alleviate changes in the condition of the scalp and hair due to temperature and humidity that change depending on the season. Furthermore, the graphene oxide has oxygen-containing functional groups attached to the edges of carbon atoms, and thus, it can combine with surrounding moisture through hydrogen bonding. Accordingly, the graphene oxide can effectively maintain moisture in the hair and scalp.

As used herein, the term “scalp or hair care” includes improving sebum, dead skin cells and troubles occurring on the scalp, and providing moisturizing and soothing effects on the scalp. In addition, it includes preventing hair loss by improving hair protection and strength, and enhancing the lubricating effect of hair (keeping hair from getting tangled).

Since the nano-carbon structure (nano-graphene oxide) prepared in the present invention significantly exhibits anti-inflammatory, non-corrosive and non-irritating skin barrier function improvements on the skin, particularly on the scalp, even in small amounts, and exhibits antibacterial effects against acne bacteria and seborrheic dandruff bacteria, it can provide excellent scalp or hair care effects, as well as prophylactic and therapeutic effects on scalp diseases.

Accordingly, in one aspect, the present invention may provide a composition for scalp or hair care, including a nano-carbon structure as an active ingredient.

In the present invention, the nano-carbon structure may be at least one selected from the group consisting of nano-graphene oxide, a nano-graphene oxide variant and graphene quantum dots.

In the present invention, the nano-graphene oxide may retain moisture through —OH groups.

In addition, nano-graphene oxide has better reactivity to functional groups than other carbon materials, thereby allowing various functional groups to be attached. The functional groups that can be attached may be polyethylene glycol (-PEG), which is a protein type, or amine group (—NH₂).

Accordingly, in the present invention, the nano-graphene oxide variant may be an aminated nano-graphene oxide or a PEGylated nano-graphene oxide.

In the present invention, the nano-carbon structure may have an average diameter of 1 to 100 nm, preferably, 5 to 75 nm, and more preferably, 5 to 50 nm.

In the present invention, the average diameter refers to the average diameter in the lateral size of graphene oxide with a certain thickness, and lateral size may be interpreted as the relatively longer length between the horizontal and vertical lengths when measuring the length of graphene oxide with a certain thickness based on a rectangular frame, or the longest distance between any two ends of graphene oxide with a certain thickness.

In the present invention, the nano-carbon structure may preferably have a thickness of 0.1 to 5 nm, and preferably, 0.5 to 3 nm.

In the present invention, the nano-carbon structure may be included at a concentration of 0.001 to 1,000 μg/mL, preferably, 0.1 to 1,000 μg/mL, more preferably, 0.1 to 100 μg/mL, and most preferably, 0.1 to 10 μg/mL. If the nano-carbon structure is included at a concentration of less than 0.001 μg/mL, the scalp or hair care effects may not appear, and if it is included at a concentration of more than 1,000 μg/mL, it may exhibit cytotoxicity.

In the present invention, the aminated nano-graphene oxide may include an amine functional group and a basic component of nano-graphene oxide at a ratio of 1:10,000 to 1:1, preferably, 1:1,000 to 1:1, and more preferably, 1:100 to 1:1.

In the present invention, the ratio is a comparison value of the number of functional groups (e.g., nitrogen) compared to the number of carbons. In NGO-NH₂, the ratio of carbon number to nitrogen number may be 5 to 20 based on nitrogen number 1.

In the present invention, the PEG functional group of the PEGylated (PEG) nano-graphene oxide may be 4arm-PEG or 6arm-PEG.

Additionally, in another aspect, the present invention may provide a pharmaceutical composition for preventing and treating a scalp disease, including a nano-carbon structure as an active ingredient.

In the present invention, the nano-carbon structure may be at least one selected from the group consisting of nano-graphene oxide, a nano-graphene oxide variant and graphene quantum dots.

In the present invention, the nano-graphene oxide variant may be an aminated nano-graphene oxide or a PEGylated nano-graphene oxide.

In the present invention, the nano-carbon structure may have an average diameter of 1 to 100 nm, preferably, 5 to 75 nm, and more preferably, 5 to 50 nm.

In the present invention, the average diameter refers to the average diameter in the lateral size of graphene oxide with a certain thickness.

In the present invention, the nano-carbon structure may preferably have a thickness of 0.1 to 5 nm, and preferably, 0.5 to 3 nm.

In the present invention, the nano-carbon structure may be included at a concentration of 0.001 to 1,000 μg/mL, preferably, 0.1 to 1000 μg/mL, more preferably, 0.1 to 100 μg/mL, and most preferably, 0.1 to 10 μg/mL. If the nano-carbon structure is included at a concentration of less than 0.001 μg/mL, it may not be effective in preventing or treating scalp diseases, and if it is included at a concentration of more than 1,000 μg/mL, it may exhibit cytotoxicity.

In the present invention, the aminated nano-graphene oxide may include an amine functional group and a basic component of nano-graphene oxide at a ratio of 1:10,000 to 1:1, preferably, 1:1,000 to 1:1, and more preferably, 1:100 to 1:1.

In the present invention, the ratio is a comparison value of the number of functional groups (e.g., nitrogen) compared to the number of carbons. In NGO-NH₂, the ratio of carbon number to nitrogen number may be 5 to 20 carbon number based on nitrogen number 1.

In the present invention, the PEG functional group of the PEGylated (PEG) nano-graphene oxide may be 4arm-PEG or 6arm-PEG.

In the present invention, the scalp disease may be at least one selected from the group consisting of psoriasis, seborrheic dermatitis, dandruff and hair loss.

As used herein, the term “pharmaceutical composition” refers to a mixture including the nano-carbon structure of the present invention and pharmaceutically acceptable excipients such as diluents or carriers. Pharmaceutical compositions include cosmetic compositions as well as compositions for therapeutic use. According to some exemplary embodiments, provided is a method of administering a pharmaceutical composition including the composition of the present invention to a subject as needed. In some exemplary embodiments, the composition of the present invention may be administered to humans.

Although the description of the pharmaceutical compositions provided herein is principally concerned with pharmaceutical compositions intended for administration to humans, those skilled in the art will understand that such compositions are generally suitable for administration to all kinds of animals. A skilled veterinary pharmacologist has a good understanding of modifications in pharmaceutical compositions for administration to various animals, and, if necessary, can design and/or perform such modifications with mere routine experimentations.

The pharmaceutical compositions described herein may be prepared by any method known in the art of pharmacology or described hereinbelow. Generally, such manufacturing methods include associating an active ingredient with an excipient and/or one or more other auxiliary ingredients, followed by shaping and/or packaging the product into desired single-or multi-dose units, if necessary or desired.

The pharmaceutical composition of the present invention may be prepared, packaged and sold as a single unit dose and/or as a plurality of single unit doses, and/or may be sold without packaging. As used herein, the term “unit dose” refers to an individual amount of a pharmaceutical composition including a predetermined amount of an active ingredient. The amount of active ingredients is generally equal to the dosage of active ingredients administered to a subject, and/or a convenient fraction of such dosage, for example, ½ or ⅓ of the dosage.

The relative amounts of active ingredients, pharmaceutically acceptable excipients, and/or any additional ingredients in the pharmaceutical compositions of the invention will vary depending on the identity, size and/or disorder of the subject to be treated and the route by which the composition is to be administered. For example, the composition may include 0.1% to 100% (w/w) of the active ingredient.

As used herein, pharmaceutically acceptable excipients include any and all of solvent, dispersion medium, diluent, or other liquid vehicle, dispersion or suspension aid, surface active agent, tonicity agent, thickener or emulsifier, preservative, solid binder, lubricant or the like that is suitable for the purpose of a particular dosage form. A use of any conventional carrier medium is considered to be within the scope of the present invention, except that it is incompatible with a substance or its derivative, for example, by providing any unwanted biological effect or otherwise interacting with any other component of pharmaceutical compositions in a harmful manner. Pharmaceutically acceptable excipients are at least 95%, 96%, 97%, 98%, 99%, or 100% pure.

The excipients are approved for human and veterinary use. In some exemplary embodiments, the excipients are approved by the US Food and Drug Administration. In some exemplary embodiments, the excipients are pharmaceutical grade. In some exemplary embodiments, the excipients meet the standards of the United States Pharmacopoeia (USP), European Pharmacopeia (EP), British Pharmacopoeia, and/or International Pharmacopoeia (Ph. Int.).

In some exemplary embodiments, excipients are approved for human and veterinary use. In some exemplary embodiments, the excipients are approved by the US Food and Drug Administration. In some exemplary embodiments, excipients are pharmaceutical grade. In some exemplary embodiments, the excipients meet the standards of the United States Pharmacopoeia (USP), European Pharmacopeia (EP), British Pharmacopoeia, and/or International Pharmacopoeia (Ph. Int.).

Pharmaceutically acceptable excipients used in the preparation of pharmaceutical compositions include inert diluents, dispersants and/or granulizers, surface active agents and/or emulsifiers, disintegrants, binders, preservatives, buffers, lubricants, and/or oils, but are not limited thereto.

Such excipients may optionally be included in the formulations of the present invention. Excipients such as cocoa butter and suppository wax, colorants, coatings, sweeteners, flavors, and perfumes may be present in the composition at the discretion of the formulator.

Exemplary diluents include calcium carbonate, sodium carbonate, calcium phosphate, dicalcium phosphate, calcium sulfate, calcium hydrogen phosphate, sodium lactose phosphate, sucrose, cellulose, microcrystalline cellulose, kaolin, mannitol, sorbitol, inositol, sodium chloride, dried starch, corn starch, powdered sugar, and combinations thereof, but are not limited thereto.

Exemplary granulating and/or dispersing agents include potato starch, corn starch, tapioca starch, sodium starch glycolate, clay, alginic acid, guar gum, citrus pulp, agar, bentonite, cellulose and wood products, natural sponges, cation-exchange resins, calcium carbonate, silicate, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (veegum), sodium lauryl sulfate, quaternary ammonium compounds, and the combinations thereof, but are not limited thereto.

Exemplary surface active agents and/or emulsifiers include natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clay (e.g., bentonite [aluminum silicate] and veegum [magnesium aluminum silicate]), long-chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol); carbomers (e.g., carboxy polymethylene,

polyacrylic acid, acrylic acid polymers, and carboxyvinyl polymers), carrageenan, cellulose derivatives (e.g., carboxymethyl cellulose sodium, powdered cellulose, hydroxymethyl cellulose, hydroxypropyl cellulose, hydroxypropyl methyl cellulose, methyl cellulose), sorbitan fatty acid esters (e.g., polyoxyethylene sorbitan monolaurate [Tween 20], polyoxyethylene sorbitan [Tween 60], polyoxyethylene sorbitan monooleate [Tween 80], sorbitan monopalmitate [span 40], sorbitan monostearate [span 60], sorbitan tristearate [span 65], glyceryl monooleate, sorbitan monooleate [span 80]), polyoxyethylene esters (e.g., polyoxyethylene monostearate [myrz 45], polyoxyethylene hydrogenated castor oil, polyethoxylated castor oil, polyoxymethylene stearate, and solutol), sucrose fatty acid esters, polyethylene glycol fatty acid esters (e.g., cremophor), polyoxyethylene ethers (e.g., polyoxyethylene lauryl ether [Breeze 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F 68, Poloxamer 188, cetrimonium bromide, cetylpyridinium chloride, benzalkonium chloride, docusate sodium, and/or combinations thereof, but are not limited thereto.

Exemplary binders include starches (e.g., corn starch and starch paste); gelatin; sugars (e.g., sucrose, glucose, dextrose, dextrin, molasses, lactose, lactitol, mannitol); natural and synthetic gums (e.g., acacia, sodium alginate, extract of Irish moss, panwar gum, shatty gum, mucilage of isapol husks, carboxymethyl cellulose, methyl cellulose, ethyl cellulose, hydroxyethyl cellulose, hydroxypropyl cellulose, hydroxypropyl methyl cellulose, microcrystalline cellulose, cellulose acetate, poly(vinyl-pyrrolidone), magnesium aluminum silicate (veegum), and larch arabinogalactan); alginate; polyethylene oxide; polyethylene glycol; inorganic calcium salts; silicic acid; polymethacrylate; wax; water; alcohol; and combinations thereof, but are not limited thereto.

Exemplary preservatives may include antioxidants, chelating agents, antimicrobial preservatives, antifungal preservatives, alcohol preservatives, acidic preservatives, and other preservatives. Exemplary antioxidants include alpha tocopherol, ascorbic acid, ascorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite, but are not limited thereto. Exemplary chelating agents include ethylene diamine tetraacetic acid (EDTA), citric acid monohydrate, disodium edetate, dipotassium edetate, edetic acid, fumaric acid, malic acid, phosphoric acid, sodium edetate, tartaric acid, and trisodium edetate. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal, but are not limited thereto. Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid, but are not limited thereto. Exemplary alcohol preservatives include ethanol, polyethylene glycol, phenol, phenolic compounds, bisphenol, chlorobutanol, hydroxybenzoate, and phenylethyl alcohol, but are not limited thereto. Exemplary acidic preservatives include vitamin A, vitamin C, vitamin E, beta-carotene, citric acid, acetic acid, dehydroacetic acid, ascorbic acid, sorbic acid, and phytic acid, but are not limited thereto. Other preservatives include tocopherol, tocopherol acetate, deteroxime mesylate, cetrimide, butylated hydroxyanisole (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glidant Plus, Fenonib, methylparaben, Germall 115, Germaben II, Neolone, Kathon, and Euxyl, but are not limited thereto. In certain exemplary embodiments, the preservative is an anti-oxidant. In another exemplary embodiment, the preservative is a chelating agent.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and combinations thereof, but are not limited thereto.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, and combinations thereof, but are not limited thereto.

Exemplary oils include almond, apricot kernel, avocado, babassu palm, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, pumpkin, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oil, but are not limited thereto. Exemplary oils include butyl stearate, caprylic triglyceride, capric triglyceride, cyclomethicone, diethyl sebacate, dimethicone 360, isopropyl myristate, mineral oil, octyldodecanol, oleyl alcohol, silicone oil, and combinations thereof, but are not limited thereto.

Liquid dosage forms for oral and parenteral administration include pharmaceutically acceptable emulsions, microemulsions, solutions, suspensions, syrups, and elixirs, but are not limited thereto. In addition to active ingredients, the liquid dosage forms may also include inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (particularly, cotton seed, groundnut, corn, germ, olive, castor, and sesame oils), glycerol, tetrahydrofurfuryl alcohol, polyethylene glycols and fatty acid esters of sorbitan, and mixtures thereof. Besides inert diluents, oral compositions may include adjuvants such as wetting agents, emulsifying and suspending agents, sweetening agents, flavoring agents, and perfuming agents. In certain exemplary embodiments for parenteral administration, the chimeric compounds of the present invention are mixed with solubilizing agents such as Cremophor, alcohols, oils, modified oils, glycols, polysorbates, cyclodextrins, polymers, and combinations thereof.

In order to prolong the effect of a drug, it is often desirable to slow the absorption of the drug from subcutaneous or intramuscular injection. This is accomplished by the use of a liquid suspension of crystalline or amorphous material with poor water solubility. The rate of absorption of the drug then depends upon the rate of dissolution which, in turn, may depend upon crystal size and crystalline form. Alternatively, the delayed absorption of a parenterally administered drug is accomplished by dissolving or suspending the drug in an oil vehicle.

Dosage forms for topical and/or transdermal administration of the nano-carbon structure of the present invention may also include ointments, pastes, creams, lotions, gels, powders, solutions, sprays, inhalants and/or patches. Generally, the active ingredient is mixed, under sterile conditions, with a pharmaceutically acceptable carrier and/or any necessary preservatives and/or buffers that may be required. Additionally, the present invention contemplates the use of transdermal patches which often have additional advantages of providing controlled delivery of active ingredients to the body. Such dosage forms can be prepared, for example, by dissolving and/or dispersing the active ingredient in a suitable medium. Alternatively or additionally, the rate may be controlled by providing a rate controlling membrane and/or by dispersing the active ingredient in a polymer matrix and/or gel.

Formulations for topical administration include liquid and/or semi-liquid preparations such as liniments, lotions, oil in water and/or water in oil emulsions such as creams, ointments and/or pastes, and/or solutions and/or suspensions, but are not limited thereto. Although the concentration of the active ingredient may be as high as the solubility limit of the active ingredient in the solvent, topically-administrable formulations may include, for example, about 1% to about 10% (w/w) of the active ingredient. Formulations for topical administration may further include one or more of the additional ingredients described herein.

The nano-carbon structure of the present invention described herein ais typically prepared in dosage unit form for easy and uniform administration. It will be appreciated, however, that the total daily usage of the composition of the present invention may be determined by the attending physician within the scope of sound medical judgment. The specific therapeutically effective dose level for any particular subject depends on a variety of factors including disease, disorder, or disorder being treated and the severity of the disorder; the activity of the specific active ingredient employed; the specific composition employed; the subject's age, weight, general health, gender and diet; the administration time, administration route, and excretion rate of the specific active ingredient employed; duration of treatment; drugs used in combination or coincidental with the specific active ingredient employed; and factors well known in the medical arts.

In certain exemplary embodiments, the pharmaceutical composition including the nano-carbon structure of the present invention may be administered daily at a dosage level sufficient to deliver about 0.001 mg/kg to about 100 mg/kg, about 0.01 mg/kg to about 50 mg/kg, about 0.1 mg/kg to about 40 mg/kg, about 0.5 mg/kg to about 30 mg/kg, about 0.01 mg/kg to about 10 mg/kg, about 0.1 mg/kg to about 10 mg/kg, or about 1 mg/kg to about 25 mg/kg of body weight of a subject at least once a day to obtain the desired therapeutic effect. The desired dosage may be delivered three times a day, twice a day, every day, every two days, every three days, every week, every two weeks, every three weeks, or every four weeks. In certain exemplary embodiments, the desired dosage may be delivered via multiple administrations (e.g., 2, 3, 4, 5, 6, 7, 8, 9, 10, 11, 12, 13, 14 or more administrations).

It will be appreciated that the dose ranges described herein provide guidance for the administration of pharmaceutical compositions provided to an adult. For example, the amount to be administered to a child or adolescent can be determined by a physician or a person skilled in the art, and may be less than or equal to that administered to an adult. The exact amount of a nano-carbon structure according to the present invention required to achieve an effective amount will vary from subject to subject, for example, depending on the subject's species, age, and overall disorder, severity of side effects or disorders, identity of the particular compound, mode of administration and the like.

It will be appreciated that the pharmaceutical composition including the nano-carbon structure of the present invention may be used in combination therapy. The particular combination of treatments (therapeutic agents or procedures) to be used in combination therapy will take into account the desired therapeutic effect to be achieved and the suitability of the desired therapeutic agent and/or procedure.

The pharmaceutical composition of the present invention may be administered alone or in combination with one or more therapeutically active agents. In the case of “combination”, although the following delivery method falls within the scope of the present invention, it is not intended to imply that the agents must be administered at the same time and/or formulated to be delivered together. The composition may be administered concurrently with, prior to, or after one or more other desired therapeutic agents or medical procedures. Generally, each agent will be administered at the dosage and/or time schedule defined for that agent. Additionally, the present invention encompasses the delivery of the pharmaceutical composition of the present invention in combination with an agent that is capable of improving the bioavailability, reducing and/or modifying the metabolism, inhibiting the secretion, and/or modifying the distribution in the body. It will be further appreciated that the nano-carbon structure of the present invention and the therapeutically active agent used in this combination can be administered together in a single composition or separately in different compositions.

The particular combination used in combination therapy will take into account the desired therapeutic effect to be achieved and/or the procedure including the nano-carbon structure of the present invention and/or the suitability of therapeutically active agent. It will be appreciated that the combination used can achieve the desired effect for the same disorder (e.g., the nano-carbon structure of the present invention may be administered in combination with another therapeutically active agent used to treat the same disorder), and/or can achieve different effects (e.g., control of any side effects).

As used herein, the term “therapeutically active agent” refers to any substance used as a medicament to treat, prevent, delay, reduce or ameliorate a disorder, and refers to a substance used in therapy, including prophylactic and curative treatments.

In some exemplary embodiments, the pharmaceutical composition of the present invention may be administered in combination with any therapeutically active agent or procedure (e.g., surgery, radiation therapy) which is useful for treating, mitigating, ameliorating, alleviating, delaying the onset of, inhibiting the progression of, reducing the severity of, and/or reducing the incidence of one or more symptoms or features of scalp diseases.

In addition, the present invention provides a method for treating a scalp disease, including the step of administering a nano-carbon structure to a patient with a scalp disease.

Since the nano-carbon structure is the same as the nano-carbon structure used in the scalp or hair care composition, the description thereof is replaced with the above description.

The scalp disease may be at least one selected from the group consisting of psoriasis, seborrheic dermatitis, dandruff and hair loss.

In addition, the present invention provides the use of a nano-carbon structure in the treatment of a scalp disease.

Since the nano-carbon structure is the same as the nano-carbon structure used in the scalp or hair care composition, the description thereof is replaced with the above description.

The scalp disease may be at least one selected from the group consisting of psoriasis, seborrheic dermatitis, dandruff and hair loss.

### Advantageous Effects

The nano-carbon structure of the present invention (e.g., aminated nano-graphene oxide and PEGylated nano-graphene oxide) is absorbed into the keratin surface of the scalp even in small amounts and affects skin cells that cause inflammation and psoriasis of the scalp, and thus, it is possible to prevent scalp diseases.

In addition, the nano-carbon structure of the present invention can induce hair growth and prevent hair loss by strengthening pores, hair roots or hair even in small amounts, and has lubricating properties that prevent hair from becoming tangled, and thus, it can be utilized in scalp and hair care products.

Furthermore, the present invention has the advantage that dispersibility problems do not occur even with proteins and other mixtures by using a nano-carbon structure with maximized dispersibility in order to increase the scalp and hair care effects.

### DESCRIPTION OF DRAWINGS

FIG. 1 shows that the nano-carbon structure of the present invention is in the shape of a coin with a size of 6 nm, as a result of photographing by using a TEM method.

FIG. 2 relates to the results of analyzing the nano-carbon structure using X-ray diffractometer (XRD).

FIG. 3 relates to the results of analyzing the nano-carbon structure using X-ray Photoelectron Spectroscopy (XPS).

FIG. 4 relates to the results of Raman analysis of the nano-carbon structure.

FIG. 5 relates to the results of analyzing the nano-carbon structure using Fourier Transform Infra-red (FTIR) spectroscopy.

FIG. 6 relates to the results of analyzing the nano-carbon structure by Atomic Force Microscopy (AFM) method.

FIG. 7 relates to the results of analyzing the nano-carbon structure using a particle size analysis method (laser diffraction method).

FIG. 8 relates to the results of analyzing the PEGylated nano-carbon structure using FT-IR analysis.

FIG. 9 relates to the results of analyzing the aminated nano-carbon structure using FT-IR analysis.

FIG. 10 shows the results of controlling the Zeta Potential of the interface of the graphene monolayer of the nano-carbon structure of the present invention to −50 mV or more.

FIG. 11 shows that, in the case of the controlled nano-carbon structure of the present invention, no agglomeration occurred in the solution state up to a concentration of 7,000 μg/mL (0.7 wt %), whereas agglomeration occurred in the solution state of the uncontrolled conventional nano-carbon structure.

FIG. 12 shows the results of confirming the cytotoxicity of the nano-carbon structure and its variants ((NGO-PEG and NGO-NH 2) of the present invention. The dotted line represents the cut-off.

FIG. 13 relates to the effect of nano-carbon structures on the differentiation rate of T helper 2 cells that cause immune responses in skin inflammation.

FIG. 14 relates to the effect of nano-carbon structures on the expression of IL-8 in keratinocytes induced by inflammation.

FIG. 15 shows the cell viability when the nano-carbon structure of the present invention was applied to an artificial skin model for 3 minutes in a skin corrosion test. The dotted line represents the cut-off.

FIG. 16 shows the cell viability when the nano-carbon structure of the present invention was applied to an artificial skin model for 1 hour in a skin corrosion test. The dotted line represents the cut-off.

FIG. 17 shows the cell viability when the nano-carbon structure of the present invention was applied to an artificial skin model in a skin irritation test. The dotted line represents the cut-off.

FIG. 18 shows the transepithelial electrical resistance (TEER) when the nano-carbon structure and carbon structure of the present invention were applied to an artificial skin model in an evaluation of the efficacy of improving skin barrier function.

FIG. 19 shows the transepidermal water loss (TEWL) when the nano-carbon structure and carbon structure of the present invention were applied to an artificial skin model in an evaluation of the efficacy of improving skin barrier function.

FIG. 20 shows the H&E staining results when the nano-carbon structure and carbon structure of the present invention were applied to an artificial skin model in an evaluation of the efficacy of improving skin barrier function.

FIG. 21 shows the antibacterial effect of the nano-carbon structure of the present invention against acne bacteria of Cutibacterium acnes.

FIG. 22 shows the antibacterial effect of the nano-carbon structure of the present invention against seborrheic dandruff bacteria of Malassezia furfur.

### MODES OF THE INVENTION

### Example 1

### Fabrication of Nano-Carbon Structure and Variant Thereof

### <1-1> Fabrication of Nano-Carbon Structure

Graphite (Qingdao Kropfmuehl, China) was used as a carbon structure, and a nano-carbon structure (nano-graphene oxide) was synthesized through the Tayler Couette flow method. The method of forming a nano-carbon structure is as follows. Graphite (graphite) is mixed with sulfuric acid (H₂SO₄) at a ratio of 1:40 to 70, and sodium nitrate (NaNO₃) is mixed with graphite (graphite) at a ratio of 1:0.2 to 0.7. After mixing the mixture at a constant rotation speed for a certain period of time, potassium permanganate (KMnO₄) is mixed at a ratio of 1:5 to 8, and then fabricated by applying a rotational force of 150 rpm to 550 rpm for 1 to 72 hours. In this case, the conditions and equipment for applying the rotational force depend on the quality of graphene oxide. The graphene oxide fabricated by the above process is nano-sized through a physical process. The nano-sized conditions vary depending on the size of the graphene oxide being nano-sized.

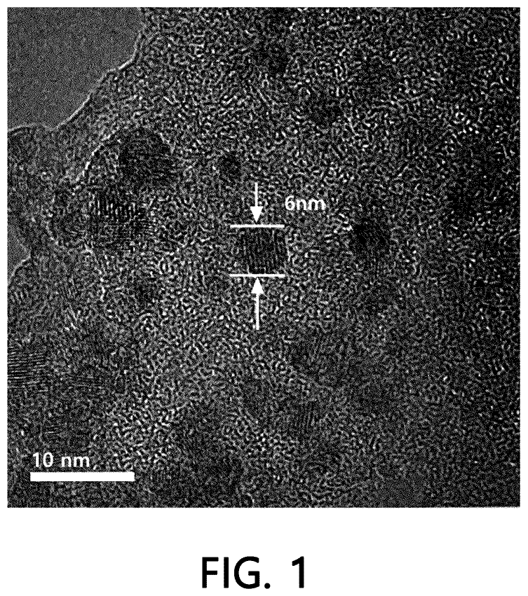

In order to prepare a nano-sized carbon structure, the obtained carbon structure is nano-sized through the following process. A solvent is added to the obtained graphene oxide to strengthen the frictional force. The commonly used solvent is DI Water, but it is set to suit the purpose of nano-sized graphene oxide. Depending on the intended use, various solvents such as ethanol, acetone and DMF may be used. The ratio of solvent used can be from the same amount to 1,000 times that of graphene oxide. This may vary depending on the nanoscale characteristics of graphene oxide. A certain amount of graphene oxide mixed in a solvent is introduced into equipment that uses a physical method to reduce the particle size of the graphene oxide using a physical method. In addition, the ratio of each material applied when fabricating graphene oxide can act as a ratio that promotes the nanonization of graphene oxide. In order to further separate the nano-sized material through physical methods in the equipment, nano-graphene oxide can be separated through a filter. In addition, it may be separated by size through a centrifugal process. In this case, the centrifuge may be used by applying the number of rotations depending on the size. When the nano-graphene oxide prepared in this way was photographed and confirmed by using the TEM method, it was found to be in the shape of a coin with a size of 6 nm (FIG. 1).

### <1-2> Fabrication of Variant of Nano-Carbon Structure

With regard to the fabrication of PEGylated nano-graphene oxide, in order to attach graphene oxide to PEG, PEG is mixed with graphene oxide 1 at a ratio of 1 (dispersed in DI with a graphene oxide concentration of 0.1 to 1 wt %): 1 to 3. (PEG Powder ratio) and mixed at room temperature for 5 to 60 minutes. In this case, the time varies depending on the amount. 5 to 20% (weight ratio) of EDC-HCl is added to the mixture and mixed at room temperature for 10 minutes to 2 hours. The rotational force applied at this time may be applied from 150 rpm to 350 rpm.

After the above process is completed, EDC-HCl is additionally added to 30 to 60% (weight ratio) of the PEG powder and mixed for 12 to 24 hours. In this case, the rotational force proceeds under the same conditions as above.

After the reaction is completed, the non-reacting solvent is removed from the mixture through high-speed centrifugation or a filter, and then, a slurry-type material is used. In case of high-speed centrifugation, material suspended at the top is used. When graphene oxide and PEG react, it becomes hydrophilic and has the property of floating in DI, and thus, when applying centrifugation, the upper material is used.

With regard to the fabrication of aminated nano-graphene oxide, in order to aminate graphene oxide, graphene oxide dispersed in DI and sodium hydrogen sulfate are mixed at a ratio of 1 (graphene oxide dispersed in DI—graphene oxide dispersed up to a concentration of 0.01 to 1.0 by weight ratio): 0.001 to 0.1 (sodium hydrogen sulfate) and mixed for 30 minutes to 4 hours. The time depends on the mixing ratio. The above mixed mixture is mixed with ammonia water at a ratio (weight ratio) of 1 (graphene oxide dispersed in DI): 0.1 to 0.5 (ammonia water with a concentration of 25 to 30%) and heated to 150° C. in a high-pressure reactor to proceed a process of reacting graphene oxide with ammonia water to attach an amine group to graphene oxide. In this case, conditions must be set such that the pressure of the high-pressure reactor does not exceed a maximum of 20 bar. The time required for the reaction varies depending on the amount, but ranges from 12 to 24 hours. The process is completed by filtering the reacted mixture through a filter with 0.22 mm pores to remove unreacted sodium hydrogen sulfate and ammonia water.

### Example 2

### Confirmation of Structural and Chemical Properties of Nano-Carbon Structure

The structural and chemical properties of each nano-carbon structure were verified through X-ray diffractometer (XRD), X-ray Photoelectron Spectroscopy (XPS), Raman analysis and Fourier-transform infrared spectroscopy (FT-IR), and the type of functional group and bond were confirmed. X-ray diffraction (XRD) is a technique that can confirm the structural information of materials in the laboratory, such as chemical composition, crystal structure, crystalline size, strain, preferred orientation and layer thickness, and X-ray photoelectron spectroscopy (XPS) is a sensitive quantitative spectroscopic technique based on the photoelectric effect that can identify elements present within a material or covering its surface, as well as the overall electronic structure and density within a material.

Furthermore, a precise analysis of the microstructure and shape of the nano-carbon structure was performed through the Atomic Force Microscope (AFM) method, and the nano size and precision were confirmed through a particle size analysis method (laser diffraction method).

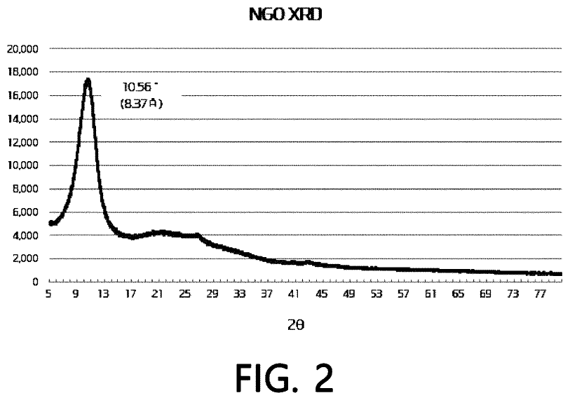

As a result of X-ray diffractometer (XRD) analysis, it was confirmed that the nano-carbon structure in the present invention was formed by splitting graphene oxide into small particles of 30 nm or less using high energy, and thus, a peak was formed at around 10° (10.56°), which is similar to the 2θ value fundamentally found in graphene oxide (FIG. 2).

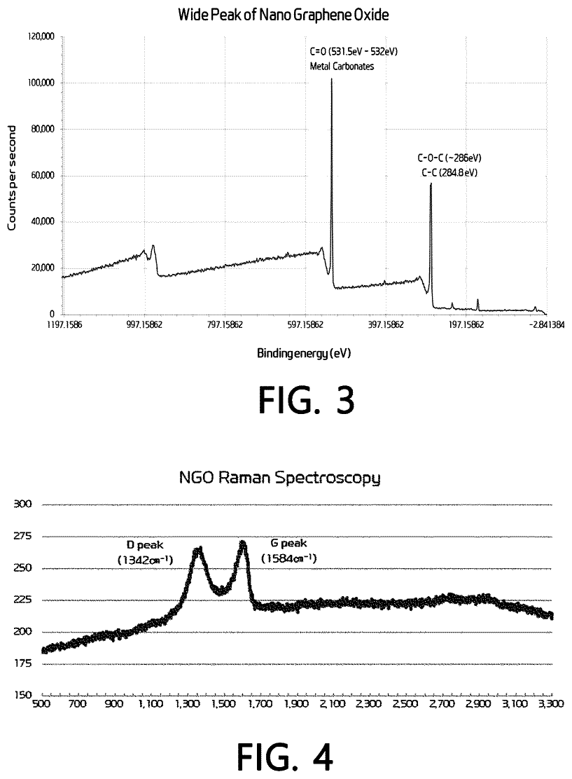

As a result of X-ray Photoelectron Spectroscopy (XPS) analysis, it was confirmed that the XPS wide peaks of the nano-carbon structure represented C═O, C—O—C and C—C bonds, respectively, and oxygen accounted for approximately 30%/weight (FIG. 3).

As a result of Raman analysis, it was confirmed that among the major peaks D, G and 2D of graphene, the D peak was formed at 1,342 cm⁻¹ and the G peak was formed at 1,584 cm⁻¹ (FIG. 4). The nano-carbon structure of the present invention was prepared in a small size by using strong energy. As the particle size gets smaller, the disordered surface structure increases, and thus, it can be confirmed that the ratio of ID/IG gradually increases. In addition, the ID/IG ratio of the nano-carbon structure shows a value of 0.98.

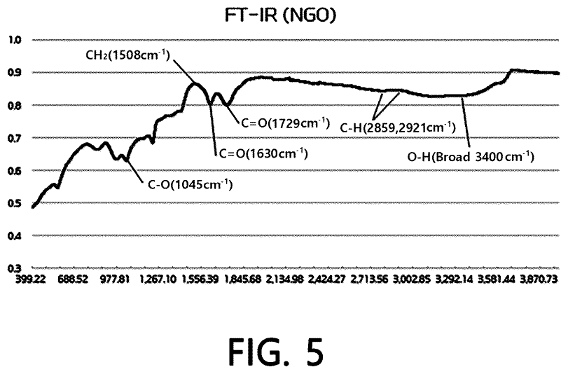

As a result of Fourier-transform infrared spectroscopy (FT-IR) analysis, the FT-IR peaks of the nano-carbon structure of the present invention were observed at CO (1,045 cm⁻¹), C═O (1,630 cm⁻¹, 1,729 cm⁻¹) and C—H (2,859 cm⁻¹) (FIG. 5). In addition, it was confirmed that the O—H Peak was boarded by damage due to high energy during the nanonization process (FIG. 5).

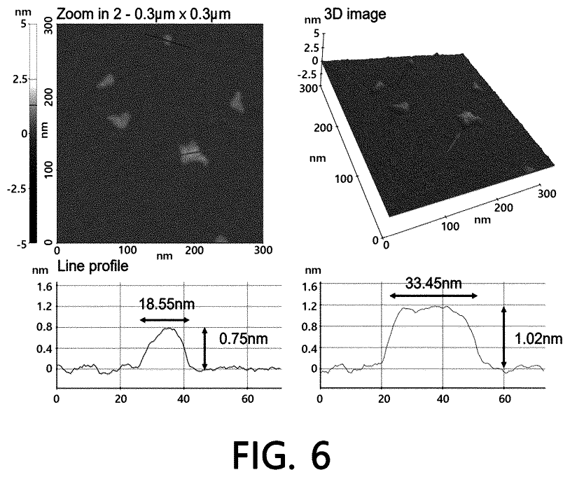

Furthermore, as a result of analysis by the Atomic Force Microscopy (AFM), it was confirmed that the particle size of the nano-carbon structure was less than 35 nm in diameter, the thickness of the nano-carbon structure was 0.34 nm, and the gap between layers was 0.7 nm. When the gap between layers measured by AFM is about 1.2 nm or less, it is a single layer. Therefore, the area and number of layers of the nano-carbon structure of the present invention were minimized for nanonization, and it was confirmed that it was mostly composed of a single layer (FIG. 6).

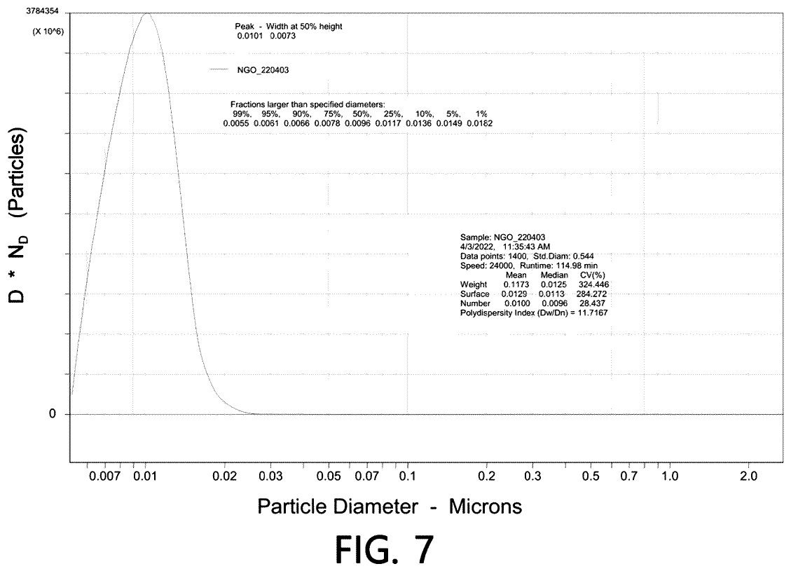

As a result of analysis by a particle size analysis method (laser diffraction method), the particle size of the nano-carbon structure was confirmed to be less than 35 nm as measured with more than 20,000 parameters, and the average particle size was 16 nm (FIG. 7).

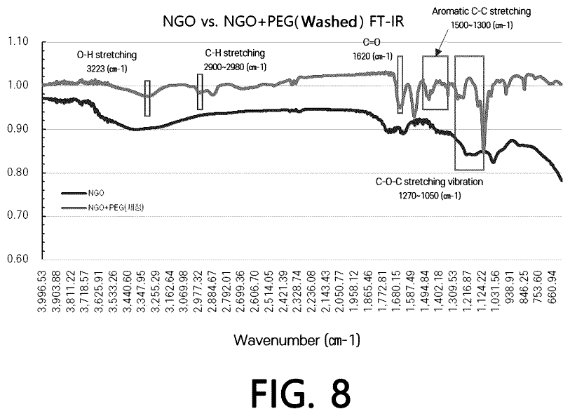

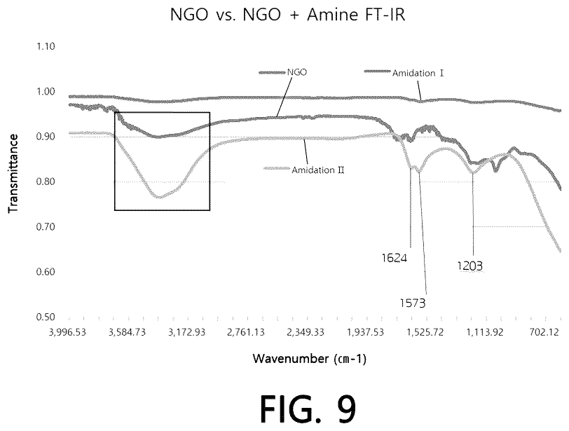

As a nano-carbon structure, in addition to nano-graphene oxide, a nano-graphene oxide variant was fabricated, and the properties thereof were confirmed by using FT-IR analysis. The characteristics of PEGylated nano-graphene oxide were determined through peaks seen in typical PEG, which do not appear in nano-graphene oxide (FIG. 8). In addition, it was confirmed that a typical amide peak was formed in aminated nano-graphene oxide (FIG. 9).

### Example 3

### Dispersion Technique for Nano-Carbon Structure

The nano-carbon structure of the present invention is graphene oxide prepared by a chemical exfoliation method. In this case, the required technique is not only a technique to control the size of several to tens of nanometers, but also a technique to disperse nano-graphene oxide in a solvent. Graphene oxide basically exists dispersed rather than dissolved in a solvent. Graphene oxide has the characteristics of high surface chemical activity and low sheet resistance, which are dominated by attractive forces such as van der Waals forces and π-π interaction, resulting in the single-layer structure of graphene oxide. A side effect of agglomeration of the laminated structure occurs.

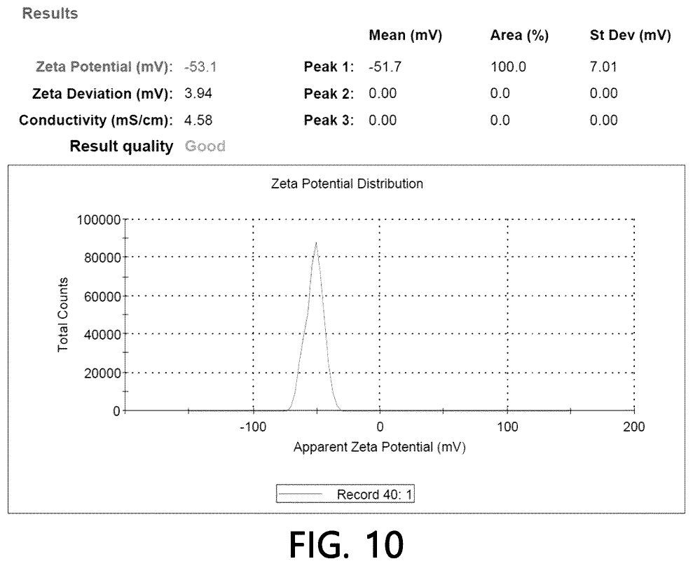

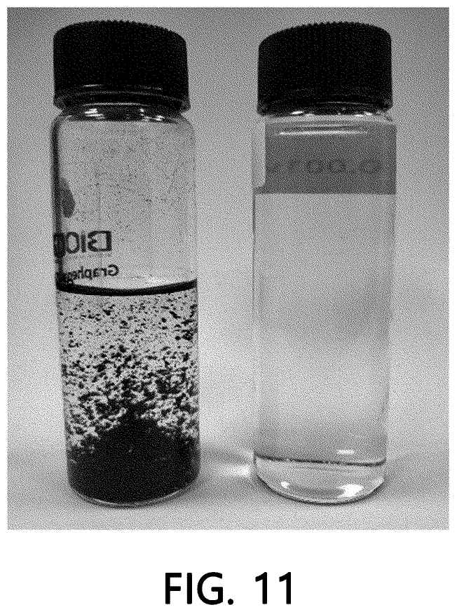

In the present invention, in order to solve this problem, the high chemical activity of the surface of the carbon nanostructure was adjusted to control the surface charge (Zeta Potential) of the interface of the graphene monolayer to more than −50 mV (FIG. 10). It was confirmed that the agglomeration that previously occurred in the solution state of the carbon nanostructure controlled in this way did not occur up to a concentration of 7,000 μg/mL (0.7 wt %) (FIG. 11).

### Example 4

### Cytotoxicity Evaluation of Nano-Carbon Structure

In order to compare and confirm the cytotoxicity of carbon nanostructures, the viability was measured at various concentrations using the MTT method.

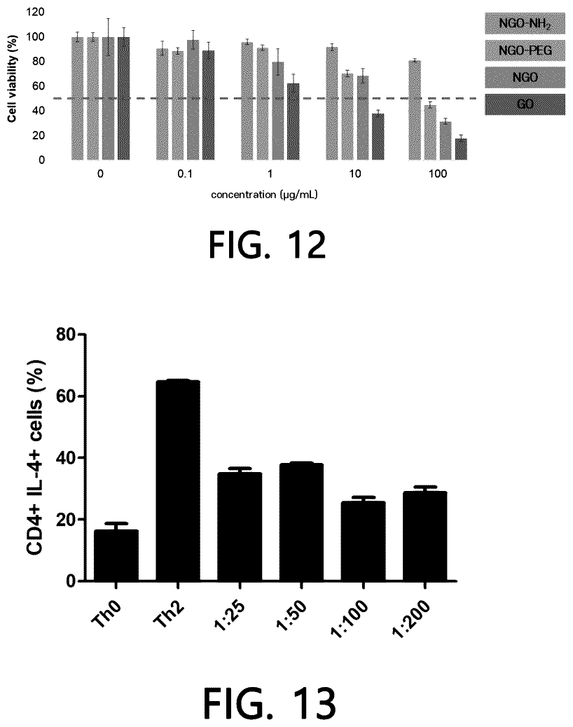

First of all, the cell viability was compared after reacting the carbon nanostructure to a mouse breast cancer cell line (4T1) for 24 hours. Nano-graphene oxide (NGO) and functionalized nano-graphene oxide (NGO-PEG and NGO-NH₂) showed cell viabilities of more than 50% up to a concentration of 10 μg/mL. However, in general graphene oxide (GO), which is not nano-sized, the cell viability decreased rapidly starting from a concentration of 1 μg/mL, and it was confirmed that the cell viability dropped to less than 30% at a concentration of 10 μg/mL. In other words, it was confirmed that cytotoxicity occurred, and it was not biocompatible (FIG. 12).

### Example 5

### Confirmation of Inhibition of T Helper 2 Cell Development by Nano-Carbon Structure

T helper 2 cells (Th 2) are known to be the main cells that cause immune responses in skin inflammation. T helper 2 cells express the GATA3 (GATA binding protein 3) transcription factor and produce IL-4 through IL-10 signaling. In the process of differentiating hPBMC (Human Peripheral Blood Mononuclear Cells; Lonza, Switzerland) into T helper 2 cells, the nano-carbon structure was treated to determine the differentiation rate of the T helper 2 cells. Specifically, after isolating mononuclear cells from cord blood, positive selection was performed immediately by using CD4 microbeads, and negative depletion was performed by using MHC class II anti-B220, F4/80 antibodies to isolate naive T cells (Th0). For T helper cell polarization, CD4+ cells (T helper cells) were isolated by using the human CD4+T Cell Isolation Kit (Miltenyi Biotec). Next, 5×10\hat{ }5 CD4+ cells were seeded per well in a 24-well plate. The culture medium was RPMI 1640 media (0.5 to 1 mL/well; Gibco), and it was cultured for 5 days under the conditions below. In this case, in order to induce CD4+ IL-4+ cells, rhIL-2 20 ng/mL, rhIL-4 20 ng/ml, and rhIL-6 20 ng/ml additives were used, and Dynabeads Human T-Activator CD3/CD28 (1:50) was added. In this case, an experimental group to which the above additives and nano-carbon structure were added and a negative control group to which only the additives were added were divided into two groups and cultured for 5 days, and the experiment was conducted.

As a result, whereas the differentiation rate of T helper 2 cells that cause skin inflammation in the negative control group (naive T cell) was about 20%, it was confirmed that it was increased to 60% or more in the positive control group (T helper 2 cell) after T helper 2 cell polarization (FIG. 13). On the other hand, in the experimental group treated with the nano-carbon structure, it was confirmed that the differentiation rate of T helper 2 cells, which cause skin inflammation, was significantly reduced to less than 40% (FIG. 13). The differentiation rate of T helper 2 cells was found to decrease at all four different graphene concentrations (1:25, 1:50, 1:100 and 1:200). In other words, the nano-carbon structure can significantly inhibit differentiation into T helper 2 cells and the development of T helper 2 cells, which cause skin inflammation.

### Example 6

### Confirmation of Decreased IL-8 Expression Level by Nano-Carbon Structure

In order to determine the effect of the nano-carbon structure on seborrheic dermatitis, the following experiment was conducted by using HaCat cells, which are a keratinocyte cell line that accounts for about 80% of the epidermis. HaCat cells were cultured in DMEM high glucose culture medium by adding 10% FBS. In this case, the confluency of the cells used in the experiment was maintained at 70% or less. In order to simulate the overexpression situation of inflammatory cytokines (post-inflammatory cytokines), cells were stimulated by treating the culture medium with IFN-gamma (10 ng/mL) and TNF-alpha (10 ng/mL) for 24 hours. In this experiment, a group of untreated cells was used as a negative control, and a group of cells treated with stimulation conditions was used as a positive control. One day after treating the irritant and controls with the nano-carbon structure, the expression level of IL-8 (interleukin-8), which is a post-inflammatory cytokine expressed in cells, was measured by using real-time RT-PCR technique. The IL-8 gene mRNA expression level was compared for each experimental group, and the results were analyzed as relative gene expression level.

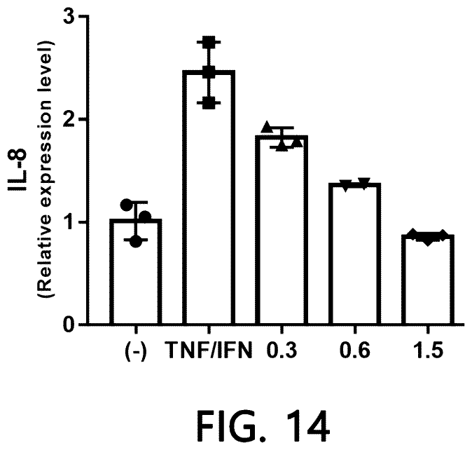

As a result, the expression level of IL-8, which is a post-inflammatory cytokine, was found to increase about 2.5 times in the positive control group under stimulation conditions (FIG. 14). On the other hand, in the experimental group treated with the nano-carbon structure under stimulation conditions, the expression level of IL-8 was confirmed to decrease depending on the concentration of the treated nano-carbon structure (0.3 μg/mL, 0.6 μg/mL, 1.5 μg/mL) (FIG. 14). In particular, when treated with the nano-carbon structure at a concentration of 1.5 μg/mL, it was confirmed that the expression level of IL-8 was reduced to the same level as the negative control group that was not treated with stimulation (FIG. 14). In other words, the nano-carbon structure of the present invention can inhibit the expression of IL-8 in keratinocytes induced by inflammation such that IL-8 is expressed at a level similar to that in cells without inflammation. Furthermore, the nano-carbon structure of the present invention can effectively alleviate the inflammatory response of scalp diseases by reducing the production of inflammatory cytokines that induce immune responses that cause the symptoms of scalp diseases such as psoriasis, seborrheic dermatitis and dandruff.

### Example 7

### Skin Corrosion Test Using Artificial Skin Model of Nano-Carbon Structure

The inventors of the present invention evaluated the skin corrosion properties of nano-carbon structures by using the artificial skin model KeraSkin. KeraSkin is an artificial skin model that reproduces the skin's epithelium using human-derived skin keratinocytes and is manufactured to be similar to real skin in terms of morphology, microstructure and biomarker expression.

Specifically, the inventors of the present invention attempted to predict the possibility of skin corrosion by measuring the damage to skin cells that appear while penetrating the stratum corneum of KeraSkin after sample treatment. This experiment was conducted by using MTT, which is a reagent for measuring the cell viability of test materials, and in compliance with the Cosmetic Risk Assessment Guidelines (Ministry of Food and Drug Safety, 2013). The cell viability of a negative control material (Deionized water) was set to 100%, and when a test material was applied for 3 minutes and the cell viability was less than 50% or when it was applied for 3 minutes and the cell viability was 50% or more and when it was applied for 1 hour and the cell viability was 15% or less, it was determined as a corrosive material, and when it was applied for 3 minutes and the cell viability was less than 50%, it was determined as a non-corrosive material. When the test material was applied for 3 minutes, the average absorbance value of the negative control material was 0.890, and when the test material was applied for 1 hour, the average absorbance value of the negative control material as 0.785, and thus, appropriate test results were derived within the suggested negative control absorbance value acceptance criteria (0.7≤OD_{NC}<1.6). When the test material was applied for 3 minutes, the cell viability of the negative control material was 100%, and the cell viability of the positive control material (8N KOH, Potassium hydroxide) was 1.6%, and when the test material was applied for 1 hour, the cell viability of the negative control material is 100.0%, and the cell viability of the positive control material was 0.6%, and thus, the test was performed appropriately.

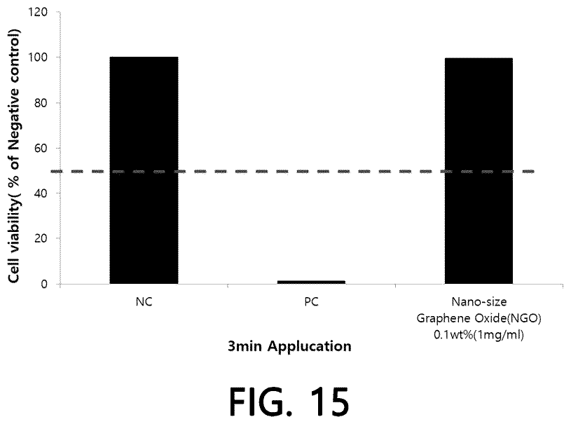

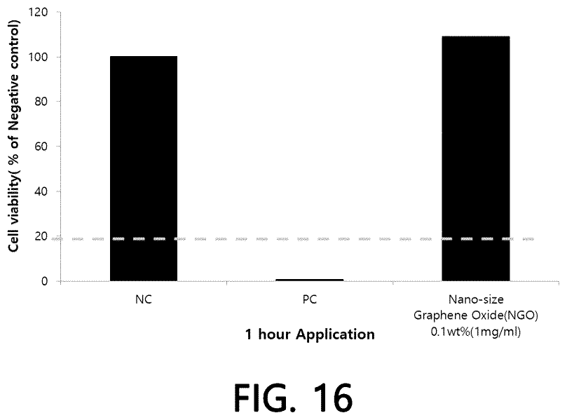

When 0.1 wt % (1 mg/mL) of the nano-carbon structure prepared in [Example 1] was applied to the artificial skin model for 3 minutes, the cell viability was 99.5% (FIG. 15), and when it was applied for 1 hour, the cell viability was 108.8%, and thus, it was evaluated as non-corrosive (FIG. 16).

### Example 8

### Skin Irritation Test Using Artificial Skin Model of Nano-Carbon Structure

The inventors of the present invention evaluated the skin irritation of nano-carbon structures using the artificial skin model KeraSkin.

Specifically, the inventors of the present invention attempted to predict the possibility of skin irritation by measuring the damage to skin cells that appear while penetrating the stratum corneum of KeraSkin after sample treatment. This experiment used MTT, which is a reagent for measuring the cell viability of test materials, and was performed in compliance with the Cosmetic Risk Assessment Guidelines (Ministry of Food and Drug Safety, 2013) and Cosmetic Toxicity Test Animal Alternative Test Method Guideline V: Skin Irritation Test Method Using a Human Skin Model (Ministry of Food and Drug Safety, 2014). The cell viability of the negative control material (DPBS, Dulbecco's Phosphate Buffered Saline) was set at 100%, and when the cell viability was greater than 50%, it was determined as a non-irritant material, and when it was less than 50%, it was determined as a skin irritant material. Since the average absorbance value of the negative control material was 1.043, appropriate test results were obtained within the suggested negative control absorbance value acceptance criteria (0.7≤OD_{NC}<1.6), and the cell viability of the negative control material was 100%, and the cell viability of the positive control material (5% SDS, sodium dodecyl sulfate) was 1.6%, and thus, the test was performed appropriately.

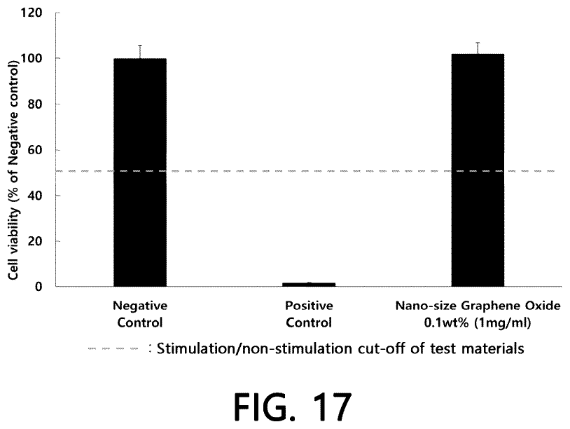

When 0.1 wt % (1 mg/mL) of the nano-carbon structure prepared in [Example 1] was applied to the artificial skin model, the cell viability was calculated to be 102.1% (FIG. 17), which was evaluated as non-irritating.

### Example 9

### Evaluation of Skin Barrier Function Improvement Efficacy Using Artificial Skin Model of Nano-Carbon Structure

The inventors of the present invention attempted to evaluate the improvement of the skin barrier function of nano-carbon structures by using the artificial skin model KeraSkin. KeraSkin is a human tissue model in which skin keratinocytes are separated from human skin tissue, amplified in vitro and cultured in three dimensions. It is structurally and functionally similar to the actual human skin epidermis. It has a multi-layer structure of basal layer, spinous layer, granular layer and stratum corneum, and expresses marker factors such as skin differentiation, stem cell function, barrier function and intercellular bonding ability (cytokeratin 10, cytokeratin 14, p63, E-cadherin, filaggrin, loricrin, involucrin).

Specifically, the test was performed in compliance with the evaluation standards for functional cosmetics presented in the Guidelines for Evaluation of Effectiveness of Functional Cosmetics (Ministry of Food and Drug Safety, 2005) and the test methods in famous academic reports related to skin research domestically and abroad. After treating the surface of KeraSkin with SDS (sodium dodecyl sulfate), which is an anionic surfactant, to weaken the skin's natural barrier function, a positive control material (10 μM retinoic acid) and a test material (0.01 wt % NGO) were added to the surface for 48 days. In order to evaluate the degree of improvement in the skin barrier function weakened by SDS by 48-hour application treatment, electrical resistance in epithelial tissue was measured by using the Millipore ERS-2 (Millipore, USA) system, and transepidermal water loss was measured by using the GPSKIN BARRIER PRO-II system, and histopathology was performed on four types of skin barrier markers through immunochemical staining.

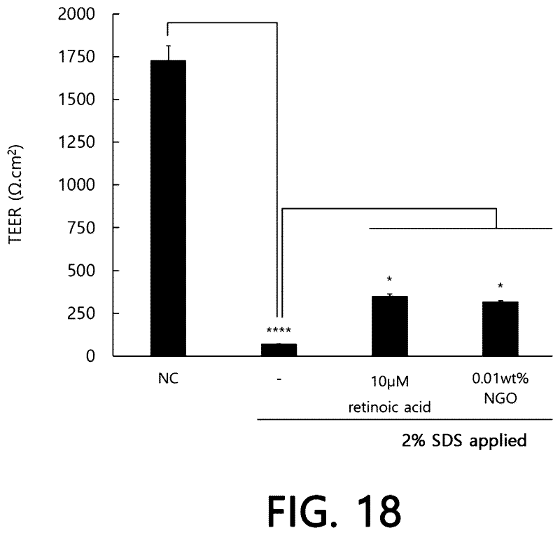

The epithelial cell layer has developed tight junctions, which are one of the forms of bonding between cells. These tight junctions form physical and chemical barriers, and the strength and integrity of these barriers can be assessed through the degree of electrical resistance across cell layers, which are called “transepithelial electrical resistance (TEER).” As the barrier function is excellent, the transepithelial electrical resistance value increases. The transepithelial electrical resistance of the normal artificial skin model was found to be 1726.8±85.3 Ω·cm², and the transepithelial electrical resistance of the barrier damaged group treated with 2% SDS was 69.2±2.1 Ω·cm², and when 10 μM retinoic acid and 0.01 wt % NGO were applied, the transepithelial electrical resistance was 347.0±15.7 Ω·cm², 313.7±10.2 Ω·cm² and 300.5±3.4 Ω·cm², respectively (FIG. 18). The barrier damage group caused by 2% SDS had a 96.0% decrease in transepithelial electrical resistance compared to the negative control group. When 10 μM retinoic acid was applied to the barrier damaged skin model, the transepithelial electrical resistance increased by 16.1% compared to the barrier damaged group. When 0.01 wt % NGO was applied to the barrier damaged skin model, the transepithelial electrical resistance increased by 14.2% and 13.4% compared to the barrier damaged group.

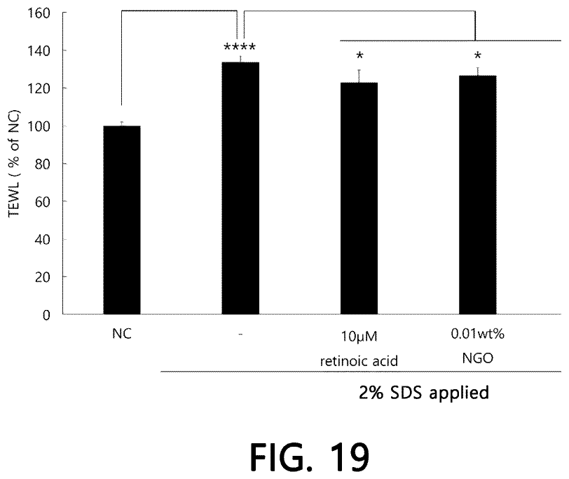

Transdermal moisture is an essential element for the skin to perform its barrier function normally, and when “transepidermal water loss (TEWL)” increases, the enzyme function required for normal exfoliation is damaged, and it is not possible to normally perform the skin barrier functions. Therefore, transepidermal water loss is used as an important physiological indicator during the damage and recovery process of the skin barrier. The transepidermal water loss of the barrier damaged group treated with 2% SDS on a normal artificial skin model increased to 133.7±3.2% compared to the negative control group, and when 10 μM retinoic acid and 0.01 wt % NGO were applied to the barrier damaged group, the transepidermal water loss rates were 122.9±6.5%, 126.6±4.0% and 123.9±3.8%, respectively, compared to the negative control group (FIG. 19). The barrier damage group caused by 2% SDS had a 33.7% increase in transepidermal water loss compared to the negative control group. When 10 μM retinoic acid was applied to the barrier damaged skin model, transepidermal water loss was reduced by 10.8% compared to the barrier damaged group. When 0.01 wt % NGO was applied to the barrier damaged skin model, transepidermal water loss was reduced by 7.1% and 9.8% compared to the barrier damaged group.

H&E staining (hematoxylin & eosin stain) is a basic staining method used in histology. Hematoxylin stains the cell nucleus blue, and eosin serves as a counterstain for areas other than the nucleus. Immunohistochemical staining is a method used to determine the expression and location of a specific antigen in tissue based on a specific antigen-antibody combination, and it is used to determine the expression and pattern of tissue pathology and skin barrier function-related factors. Among skin barrier function-related factors, filaggrin is an essential factor in regulating epidermal homeostasis and is used as a component of the lipid envelope within the stratum corneum to perform moisturizing and skin barrier functions. p63 is a protein that regulates cell proliferation and is mainly expressed in the nucleus of cells, E-cadherin is an intercellular junction protein expressed in the cell membrane as an indicator of cell-to-cell contact, and cytokeratin 14 (CK14) is a protein that forms the skeleton of epidermal cells, and is expressed in the basal layer and stratum spinosum.

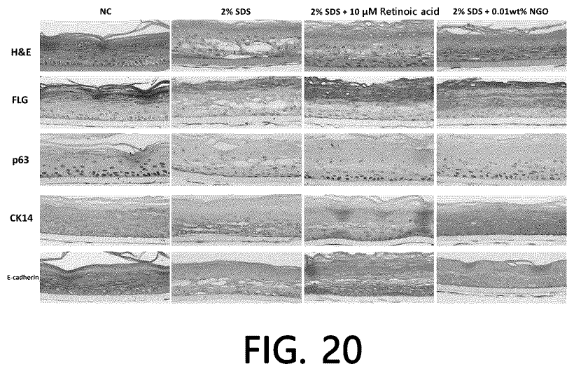

As a result of determining histopathology through H&E staining, the normal artificial skin model had close adhesion between cells and formed four differentiated layers including the stratum corneum, and when it was treated with 2% SDS, the basal layer was damaged, and vacuoles were formed between cells (FIG. 20). When 10 μM retinoic acid and 0.01 wt % NGO were applied after barrier damage with 2% SDS, it was confirmed that the recovery of the basal layer and vacuoles between cells were improved compared to the barrier damage group. As a result of determining the histopathology of filaggrin, p63, E-cadherin and CK 14 through immunohistochemical staining, filaggrin was expressed in the stratum corneum, p63 was expressed in the cell nucleus, E-cadherin was expressed in the membrane portion of the cells, and CK14 was expressed in the epidermis excluding the stratum corneum (FIG. 20). When it was treated with 2% SDS, compared to the negative control group, the expressions of filaggrin and CK14decreased, and p63 and E-cadherin were not expressed. When 10 μM retinoic acid was applied after barrier damage with 2% SDS, the expressions of filaggrin, p63, E-cadherin and CK14 all increased compared to the barrier damaged group. Even when 0.01 wt % NGO was applied, the expressions of filaggrin, p63, E-cadherin and CK14 all increased compared to the barrier damaged group.

### Example 10

### Confirmation of Antibacterial Effects of Nano-Carbon Structure

The antibacterial activity of the nano-carbon structure (0.1 wt % daNGO) of the present invention was determined against two representative strains (Cutibacterium acnes and Malassezia furfur) that are present in the skin and oral cavity.

Specifically, the acne bacteria of Cutibacterium acnes were cultured in Sheep blood TSA medium under anaerobic conditions at 37 degrees. In this experiment, the seborrheic dandruff bacteria of Malassezia furfur were cultured in mLNA medium under aerobic conditions at 30 degrees. After the nano-carbon structure was exposed to bacteria cultured for 6 days each for 48 hours, the reduction rate thereof was measured. The reduction rate was calculated as a percentage by subtracting the number of bacteria over time from the initial number of bacteria.

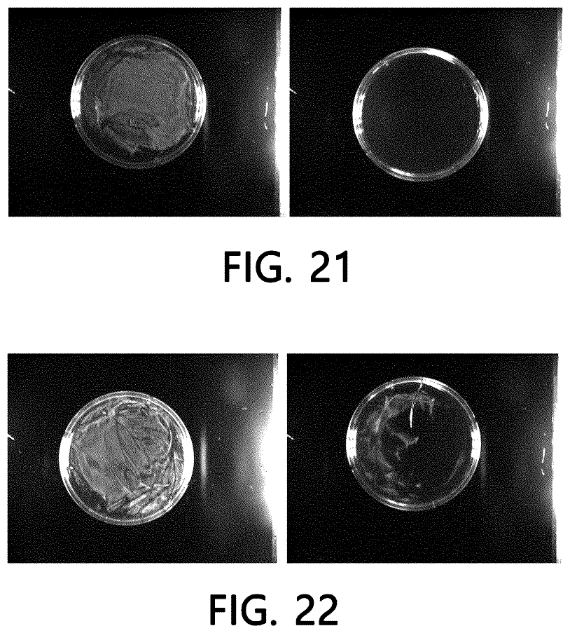

As a result, all CFU of the acne bacteria disappeared at the initial inoculation number of 8.2×10\hat{ }4 CFU/mL, and the reduction rate therefor was shown to be 99.9% (FIG. 21). Seborrheic dandruff bacteria decreased from the initial inoculation number of 3.9×10\hat{ }4 CFU/mL to 20,500 CFU/mL, and the reduction rate therefor was confirmed to be 47.4% (FIG. 22).

### INDUSTRIAL APPLICABILITY

The nano-carbon structure (nano-carbon graphene, nano-graphene oxide) prepared in the present invention significantly exhibits anti-inflammatory, non-corrosive and non-irritating skin barrier function improvements on the skin, particularly on the scalp, even in small amounts, and exhibits antibacterial effects against acne bacteria and seborrheic dandruff bacteria, and thus, since it can provide excellent scalp or hair care effects and prophylactic and therapeutic effects on scalp diseases, it has industrial applicability.

1. A composition for scalp or hair care, comprising a nano-carbon structure as an active ingredient.

2. The composition of claim 1, wherein the nano-carbon structure is at least one selected from the group consisting of nano-graphene oxide, a nano-graphene oxide variant and graphene quantum dots.

3. The composition of claim 2, wherein the nano-graphene oxide variant is an aminated nano-graphene oxide or a PEGylated nano-graphene oxide.

4. The composition of claim 1, wherein the nano-carbon structure has an average diameter of 1 to 100 nm.

5. The composition of claim 1, wherein the nano-carbon structure has a thickness of 0.1 to 5 nm.

6. The composition of claim 1, wherein the nano-carbon structure is comprised at a concentration of 0.001 to 1,000 μg/mL.

7. The composition of claim 3, wherein the aminated nano-graphene oxide comprises an amine functional group and a basic component of nano-graphene oxide at a ratio of 1:10,000 to 1:1.

8. The composition of claim 3, wherein the PEG functional group of the PEGylated nano-graphene oxide is 4arm-PEG or 6arm-PEG.

9. A method for or hair care, comprising applying a nano carbon structure to a scalp or hair.

10. The method of claim 9, wherein the nano-carbon structure is at least one selected from the group consisting of nano-graphene oxide, a nano-graphene oxide variant and graphene quantum dots.

11. The method of claim 10, wherein the nano-graphene oxide variant is an aminated nano-graphene oxide or a PEGylated nano-graphene oxide.

12. The method of claim 9, wherein the nano-carbon structure has an average diameter of 1 to 100 nm.

13. The method of claim 9, wherein the nano-carbon structure has a thickness of 0.1 to 5 nm.

14. The method of claim 9, wherein the nano-carbon structure is comprised at a concentration of 0.001 to 1,000 μg/mL.

15. The method of claim 11, wherein the aminated nano-graphene oxide comprises an amine functional group and a basic component of nano-graphene oxide at a ratio of 1:10,000 to 1:1.

16. The method of claim 11, wherein the PEG functional group of the PEGylated nano-graphene oxide is 4arm-PEG or 6arm-PEG.

17-19. (canceled)

20. A method for treating a scalp disease, comprising administering a nano-carbon structure to a patient in need thereof with a scalp disease.

21. The method of claim 20, wherein the scalp disease is at least one selected from the group consisting of psoriasis, seborrheic dermatitis, dandruff and alopecia.

22. The method of claim 20, wherein the administering comprises applying the nano-carbon structure to a scalp or hair of the patient.
